(19) United States
(12) Patent Application Publication
(10) Pub. No.: US 2013/0270459 A1
(43) Pub. Date: Oct. 17, 2013

(54) AUTONOMOUS CLEANING ROBOT

(71) Applicant: Alex Fontani, Austin, TX (US)

(72) Inventor: Alex Fontani, Austin, TX (US)

(21) Appl. No.: 13/862,973

(22) Filed: Apr. 15, 2013

Related U.S. Application Data
(60) Provisional application No. 61/624,641, filed on Apr. 16, 2012.

(51) Int. Cl. A61L 2/10

(52) U.S. Cl. CPC A61L 2/10; Y10S 901/01
USPC 250/492.1; 901/1

(57) ABSTRACT

An autonomous cleaning robot consists of a housing, the autonomous system to drive the robot, and a high power ultraviolet bulb. The ultraviolet bulb is used to disinfect the cleaning surface from various types of allergens including bacteria, mold, and dust mites. The robot itself consists of a hard plastic shell that houses the robots main components and a screen indicating battery life, and cleaning time as well as two buttons to turn the system on and off. The autonomous system uses a series of ultrasonic sensors to calculate the position of the robot relative to obstacles which allow for safe autonomous movement

### CROSS-REFERENCE TO RELATED APPLICATIONS

This application claims priority to U.S. Provisional Application Ser. No. 61/624,641, titled “AUTONOMOUS CLEANING ROBOT” with a filing date of Apr. 16, 2012, which is hereby incorporated by reference.

### BACKGROUND OF THE INVENTION

In the U.S. alone over 50 million people are affected by the allergic effects of house borne micro organisms mainly caused by the fecal waste produced by dust mites in household carpet. The traditional method for treating carpets with dust mites involves the use of water or steam based carpet cleaners, but this can be costly, time-consuming and often requires rental of specialty equipment or contracting services. In addition the carpet only stays clean for a certain amount of time before the organisms repopulate the carpet. However with the application of high power ultraviolet light these organisms can easily be killed with little effort. Ultraviolet technology has existed for many decades and has been integrated into many practical devices including hand held wands manufactured by companies such as Germ Guardian Inc., that can be used for sanitizing small surfaces such as bed spreads. However these wands are not practical for use on large areas; in addition using them can be tiresome as well as hazardous to human health because of the risk of skin cancer when exposed accidentally to skin. My invention could be used to rid household carpet of many of these organisms with out any of the disadvantages and dangers of human use of the ultraviolet technology.

The approaches described in this section could be pursued, but are not necessarily approaches that have been previously conceived or pursued. Therefore, unless otherwise indicated herein, the approaches described in this section are not prior art to the claims in this application and are not admitted to be prior art by inclusion in this section.

### BRIEF SUMMARY OF THE INVENTION

The invention utilizes a UVC bulb 6 to disinfect carpet surfaces of dust mites and other micro organisms. The invention moves at a slow rate over a carpet surface to ensure proper exposure to UVC radiation by the micro organisms to ensure eradication. The autonomous device uses ultrasonic sensors 15 to figure out its position in space, and be aware of any obstacles in its way during the sanitizing process. The robot uses a general behavior pattern program that is applied to the entire perimeter of the room. Once the entire power supply 16 of the robot has run out it emits an audible tone indicating it needs to be recharged via the two charging terminals 8. The entire hardware is operated with two simple buttons 2 to control the start and stop states of the machine, as well as a charge indicator light. An informative LCD display screen 10 informs the user total running time left until the battery is fully discharged as well as the current cleaning status of the machine. For movement across surfaces the robot utilizes two driver wheels 5 and one rear caster wheel 4. The whole machine is encased in a durable plastic casing ensuring long lasting durability.

These and other aspects of the disclosed subject matter, as well as additional novel features, will be apparent from the description provided herein. The intent of this summary is not to be a comprehensive description of the claimed subject matter, but rather to provide a short overview of some of the subject matter's functionality. Other systems, methods, features and advantages here provided will become apparent to one with skill in the art upon examination of the following FIGURES and detailed description. It is intended that all such additional systems, methods, features and advantages that are included within this description, be within the scope of any claims filed now or at a later time.

### BRIEF DESCRIPTIONS OF THE DRAWINGS

The novel features believed characteristic of the invention will be set forth in any claims that are filed later. The invention itself, however, as well as a preferred mode of use, further objectives, and advantages thereof, will best be understood by reference to the following detailed description of an illustrative embodiment when read in conjunction with the accompanying drawings, wherein:

FIG. 1 depicts a perspective view of the autonomous cleaning robot in a fully assembled condition.

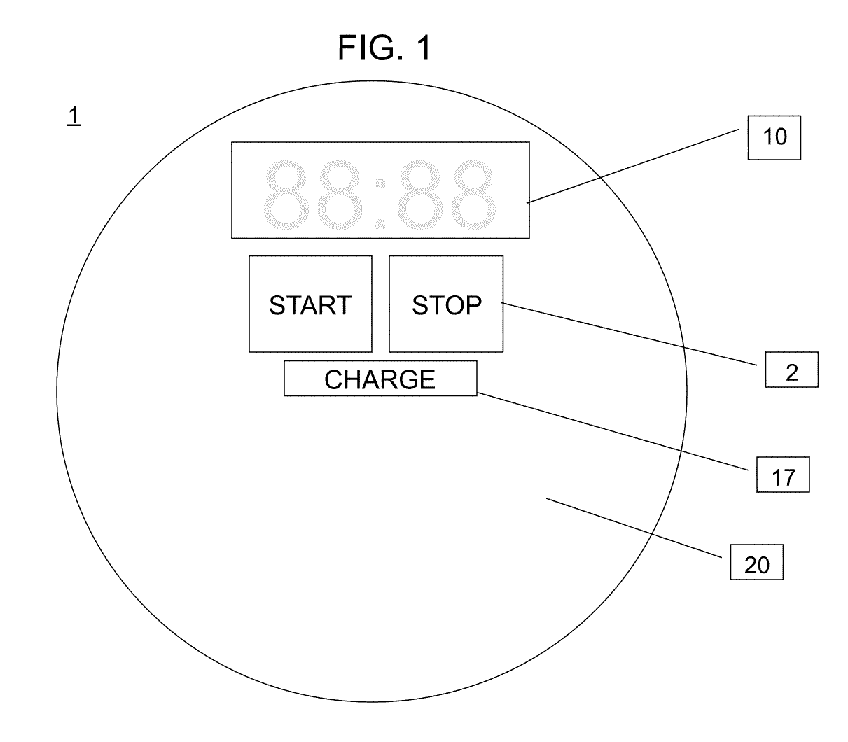

FIG. 2 depicts an underside view of the autonomous cleaning robot and its components.

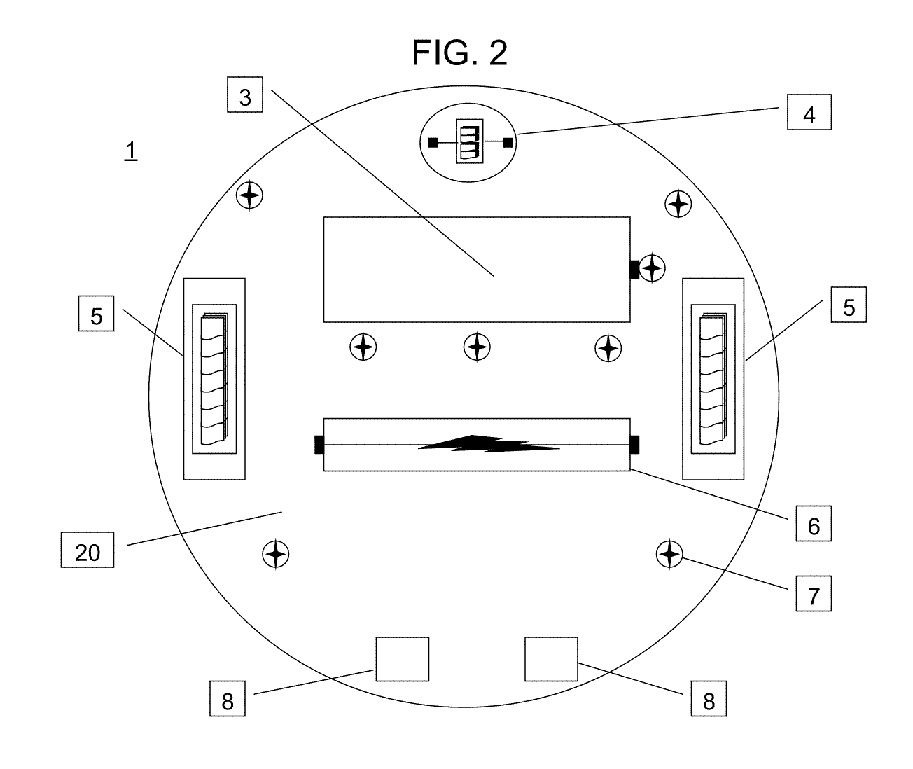

FIG. 3 depicts a cross-sectional view of the autonomous cleaning robot and its inside components.

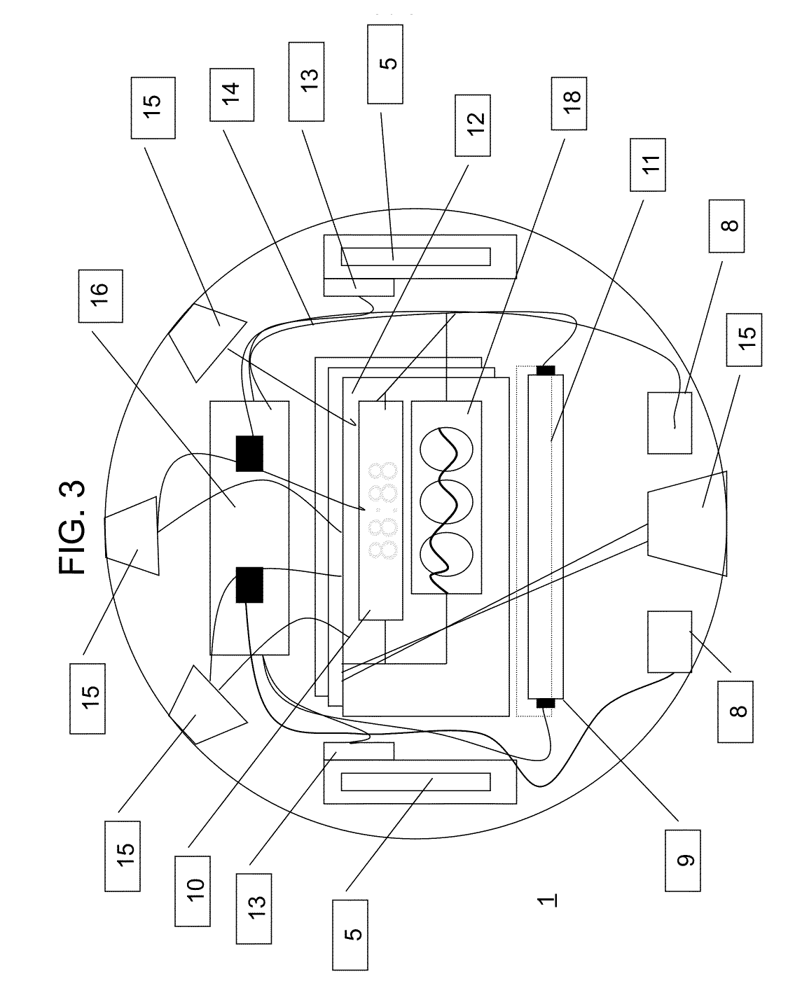

In the FIGURES, like elements should be understood to represent like elements, even though reference labels are omitted on some instances of a repeated element, for simplicity.

### DETAILED DESCRIPTION OF ILLUSTRATIVE EMBODIMENTS

Reference now should be made to the drawings, in which the same reference numbers are used throughout the different figures to designate the same components. An embodiment of the present invention includes an autonomous cleaning robot 1 which utilizes a UVC bulb 6 within a housing 20 to disinfect carpet surfaces of dust mites and other microorganisms. While some embodiments utilize an autonomous device, other embodiments may use other devices such as manually operated devices, any type of vacuum cleaner, etc. In some embodiments, the device moves over a carpet surface at a rate slow enough to ensure proper exposure to UVC radiation by the micro organisms to ensure eradication.

In some embodiments, a UVC system 11 embedded in the invention consists of a single UVC bulb 6 suspended within the housing 20, and connected to a main power supply 16. While a single UVC bulb 6 is used in the present embodiment for the purposes of disinfecting, any number of bulbs may be used, of any type of bulbs, for example UVA, etc., for other purposes. While in operation, the UVC bulb is turned on continuously, only being turned off when a temperature probe 9 detects a temperature above a predetermined normal operating range, for example, a temperature above 150°. The invention may utilize readily available commercial grade UVC lights which can be purchased from commercial distributors, for example, SOS Inc. in Dallas, Tex. or directly from manufacturers, for example, American Ultraviolet Co. The UVC bulbs emit ultraviolet radiation in a wavelength range of 100 nm to 280 nm. UV radiation in these wavelengths will effectively kill any microorganism after approximately 5 seconds of exposure.

These UVC bulbs typically require 11 volts of power to operate, but others are available at lower voltage levels, such as 6 volts. Care is taken to engineer the right level of power to the bulb, so as to prevent overheating of the bulb, leading to a reduction of the operating life of the bulb. To minimize risk and prolong bulb life, in some embodiments, the temperature probe 9 is added to turn off the bulb automatically by cutting off its power source, whenever it detects the bulb is operating above the predetermined normal temperature operating range.

Some embodiments of the invention utilize one or more ultrasonic sensors 15. The ultrasonic sensors work by emitting a high frequency tone and measuring the time it takes for them to bounce back after hitting any object, to calculate the distance of the device from that object. Any number of ultrasonic sensors can be used in any position, for example, six ultrasonic sensors spread evenly around the perimeter of the invention, etc. The continuous ultra sonic readings are sent to a programmable chip 12 which is programmed to determine the dimensions of the room, map objects within that room and at any point in time determine the location of the device in the room, plus any obstacles in the immediate vicinity of the device. This can be achieved by using, for example, a general behavior pattern program applied to the entire perimeter of the room. Other embodiments may utilize systems other than ultrasonic sensors to determine the perimeter of the room.

Once the entire power supply 16 of the robot has run out it emits one or more audible tones indicating it needs to be recharged via two charging terminals 8. Additionally, the power supply 16 can be accessed through the battery compartment 3 for replacement or otherwise. The autonomous cleaning robot 1 may automatically return to be charged or the charging terminals may be manually connected. In various embodiments, the device may be charged by any means, for example, a base station connected to a transformer which connects to an outlet, a single charging terminal, a direct connection to an outlet, a solar charge connection etc.

In some embodiments the device is operated with two simple buttons 2 to control the start and stop states of the machine, but other embodiments may include different user interfaces with any number of buttons. One or more charge indicator lights 17 may be used to indicate, for example, a full charge, a partial charge, a full discharge, a current status of charging, etc. One or more informative LCD display screens 10 may be used to inform the user, for example, of the total running time left until the power source 16 is fully discharged, the current cleaning status of the device, the expected total time to clean the current room, the status of the bulb 6, etc. Current cleaning status messages may include, for example, cleaning, low battery, charging, bulb on, bulb off, etc. Some embodiments may include different modes of cleaning, for example, spot cleaning, cleaning limited to a certain perimeter, cleaning limited by time, cleaning until the job is complete, cleaning until the power source 16 is fully discharged, etc. The cleaning mode may also be indicated on the one or more LCD display screens 10. A central printed circuit board (PCB) 18 and a plurality of connecting wires 14 allow the various components of the autonomous cleaning robot 1 to communicate. Additionally, the connecting wires 14 are used to provide power to various components of the autonomous cleaning robot 1.

For movement across surfaces some embodiments utilize two driver wheels 5 powered by driver wheel motors 13 and one rear caster wheel 4, while other embodiments may use any number of wheels in any number of positions. In some embodiments, the whole device is encased in a durable plastic housing 20 ensuring long lasting durability, while in other embodiments other materials may be used, for example, metal, composite materials, etc. While in some embodiments the housing 20 and the battery compartment 3 are fastened with a plurality of screws 7, other embodiments may use different fasteners.

While the invention has been described with respect to a limited number of embodiments, the specific features of one embodiment should not be attributed to other embodiments of the invention. No single embodiment is representative of all aspects of the inventions. Moreover, variations and modifications therefrom exist. For example, the invention described herein may comprise other components. Various additives may also be used to further enhance one or more properties. In some embodiments, the inventions are substantially free of any additive not specifically enumerated herein. Some embodiments of the invention described herein consist of or consist essentially of the enumerated components. In addition, some embodiments of the methods described herein consist of or consist essentially of the enumerated steps. The appended claims intend to cover all such variations and modifications as falling within the scope of the invention.

What is claimed is:

1. A disinfecting apparatus, said disinfecting apparatus comprising:
an autonomous robot, said autonomous robot having a top and a bottom, wherein said bottom faces a surface; and
one or more ultraviolet bulbs, wherein said one or more ultraviolet bulbs are attached to the bottom of said autonomous robot, such that said one or more ultraviolet bulbs emit ultraviolet light on the surface, disinfecting the surface, wherein said autonomous robot is capable of independently moving across the surface to disinfect the surface with said one or more ultraviolet bulbs.
